(19) United States
(12) Patent Application Publication
(10) Pub. No.: US 2008/0146991 A1
(43) Pub. Date: Jun. 19, 2008

(54) Hand operated dispenser for surgical irrigation fluids

(76) Inventors: Chris Hernandez, The Woodlands, TX (US); Tom Burick, West Chicago, IL (US)

Correspondence Address: KAMMER BROWNING PLLC, 7700 BROADWAY, SUITE 202, SAN ANTONIO, TX, 78209, US

(21) Appl. No.: 11/641,591

(22) Filed: Dec. 18, 2006

(51) Int. Cl. A61M 1/00

(52) U.S. Cl. 604/34; 604/30; 604/151; 604/247; 604/113

(57) ABSTRACT

A system for dispensing irrigation fluids, including a hand operated dispensing unit connected through a continuous length of multi-tube conduit to a mobile reservoir cart. The dispensing unit is constructed of disposable materials. The conduit system is uninterrupted to provide a sealed delivery of the irrigating fluids to the dispensing unit. Flow control is through a trigger mechanism on the handheld unit, as well as a plurality of individual on/off valves. A variety of nozzles are provided including devices for flushing and/or debriding. The reservoir unit includes disposable containers for the variety of irrigation fluids that are positioned in place and connected to the tubing conduit that extends to the handheld unit. A fluid heating unit may optionally be incorporated. Rotary tube pumps provide a nearly continuous flow of fluid and are capable of continuous operation even with back pressure due to flow stoppage at the handheld unit.

### BACKGROUND OF THE INVENTION

1. Field of the Invention

] The present invention relates generally to systems and methods for fluid irrigation utilized in conjunction with surgical and medical procedures. The present invention relates more specifically to a mobile, manually and locally controlled system for dispensing a variety of surgical irrigation fluids. 2. Description of the Related Art

] In many different surgical and medical procedures it is desirable to have available a number of different fluids for the purpose of irrigating that portion of the patient's body undergoing the surgical or medical procedure. Quickly and controllably dispensing such fluids serves a number of different purposes which benefit the overall process of carrying out the surgical or medical procedure. It is often desirable, for example, to simply rinse away body fluids that are secreted into the surgical area to facilitate the surgeon's access to the target tissues and organs. Fluid irrigation also facilitates the maintenance of the health of the tissue undergoing surgery, both in terms of maintaining a healthy fluid environment, and in rinsing away potential contaminants to the tissues.

In general, physicians have heretofore had to deal with irrigation systems that were inadequate in their capabilities or extremely cumbersome in their configuration and size. Such systems were often capable of dispensing only a single type of fluid from a single dispensing line. In the simplest form, an irrigation system might include a section of tube extending from a sterile fluid reservoir such as sterile water or sterile saline solution. Treatment of wounds in the acute setting often requires disinfecting, irrigating, and aggressive debridement. Since such a process can involve several variable factors to consider, it frequently is a tedious process. It may involve several types of solutions, the need for pulsed lavage, drainage of solutions, and often the assistance of another health care worker. Thus, a great deal of time and efficiency is lost as the primary physician must give directions to the assisting health care providers to carry out the irrigation properly according to the patient's needs and the primary surgeon's needs at the particular stage of the process.

Such simple irrigation and suction systems were typically connected to reservoirs mounted on the wall in the operating room or through the wall to common vacuum or suction sources within the hospital environment. Control of the flow of both the irrigating fluid and the suction was typically a simple on/off valve, often positioned adjacent the reservoir, or at the outlet from the wall of the operating room. The result of this configuration would be that the health care providers would have to redirect the flow of irrigating fluid out of the surgical area or back into the area as needed, rather than actually turning the flow on or off. The same was typical of providing suction or vacuum to the surgical area, although the consequences of simply redirecting the suction line out of the patient are not as significant as with an irrigation fluid flow. In any event, the process of controlling the flow of irrigation fluid, especially the simultaneous flow of a number of different types of irrigation fluid, was wholly inadequate under the system of connecting tubes or conduits to large scale reservoirs of fluid.

Some attempts have been made in the health care field to provide mechanisms for delivering the flow of irrigation fluids more effectively to the surgical area. Some of these likewise attempt similar efforts at providing suction for the removal of the irrigation fluids after use. Some of these efforts have attempted to provide hand-gripped devices that allow the primary physician some level of control over the flow of irrigation fluids and the provision of suction to the surgical area. In general, however, these past efforts have failed to address all of the needs associated with the process of effectively irrigating and controlling the flow of fluids into and out of a surgical site while remaining economically feasible. Some of the efforts in the prior art include the following:

U.S. Pat. No. 6,352,527 issued to Henniges et al. entitled Surgical/Medical Irrigator with Rechargeable Battery Pack describes a system for use in conjunction with surgical and medical procedures that includes a hand piece with a tip assembly including a drain tube and a discharge tube. This system includes a hand-gripped trigger that allows the surgeon to position the hand piece and to turn on or off the irrigator.

U.S. Pat. No. 5,147,292 issued to Kullas et al. entitled Control Handle with Locking Means for Surgical Irrigation describes a hand piece for surgical irrigation and suction with a specifically designed drip free valve arrangement that serves to kink the irrigation conduit, thereby discontinuing the flow through the conduit. The degree to which the irrigation tubing is kinked is controlled by the trigger, which can be locked into a full flow or no flow configuration.

U.S. Pat. No. 5,419,772 issued to Teitz entitled Surgical Irrigation Apparatus for Cleaning and Sterilizing Wounds in Surgical Areas During Surgery describes an irrigation system that includes an elongated hand held instrument for applying a solution spray to a surgical area. Various nozzles are selected and connected to the device to create various flow patterns at the surgical site. A pressurized liquid supply utilizing a bulb pump or small pressurized canister establishes a pressure of about 100-600 mm Hg therein. This provides an irrigating solution to the wound at about 8-15 psi.

U.S. Pat. No. 6,059,754 issued to Pasch et al. entitled Pulsed Lavage Pump with Integral Power Search and Variable Flow Control describes a self contained pulsatile surgical irrigation device that includes a trigger connected to a housing and a motor within the housing associated with the pump. The trigger is mechanically connected to the drive system to enable the stroke of the pump to be varied, thereby varying the output flow from the device.

Various other attempts have been made in the medical and dental fields to provide hand gripped dispensing devices for fluids to be delivered into surgical areas or other areas of the patient undergoing medical procedures. In general, however, these devices fail to provide the versatility required by the physician who frequently needs a multitude of different irrigating fluids, as well as different rates of delivery and different placements and positioning of suction. Most of the prior art devices additionally are associated with fixed reservoirs or vacuum sources in a manner that prevents their easy manipulation by the physician. While some of the systems described in the prior art address one or more of the various problems identified, none address all of the problems identified in a single unit.

In general, a tradeoff remains between a system that is versatile enough to provide a variety of irrigation fluids in conjunction with suction flow in a system that is not cumbersome or overly difficult to manipulate. In other words, those systems that do provide some versatility with regard to a selection of nozzles and fluids delivered from a handheld unit are capable of providing such versatility only at the sacrifice of size and convenience. Those systems that provide ease of use through size, mobility, and convenience, have generally been incapable of providing multiple fluids and versatility of flow control. Existing systems that provide the versatility of multiple fluids (and even some that dispense a single fluid) often require extensive sterilization efforts in order to make components within the system re-usable. The ability to re-use these components becomes important because of the high manufacturing costs typically associated with such. No systems in the prior art have achieved the ability to dispose of patient contact items and still re-use a portable capital item.

It would therefore be desirable to have a system that addressed each of the problems associated with irrigating surgical and medical procedure sites. It would be desirable if such a system could dispense a number of different fluids at the selection of the surgeon or physician carrying out the procedure. It would be desirable if such flow control for a multitude of fluids could be accomplished with one hand during the surgical or medical procedure so as to allow the primary physician complete and direct control over the irrigation process. It would be desirable if the system was capable of integrating a number of different nozzle structures that were easily and rapidly interchangeable, to adjust for temperature, and to allow the surgeon use of the various fluids in a number of different circumstances during the surgical or medical procedures.

It would be essential for such a handheld device to operate without the need for any electrical power at the handheld unit itself. It would be desirable if the components of the handheld unit were simple enough and easy enough to manufacture that they could be constructed into a disposable unit as opposed to requiring sterilization prior to reuse. It would be desirable if such a system included a reservoir source that was mobile instead of fixed in the structural confines of the hospital or operating room permanent fixtures. It would be desirable if such a mobile system provided direct and immediate access to the system by the attending physician. The ability to place such a system in close proximity to the patient and the attending physician would not only provide immediate fluid delivery, but also the possibility of warming the fluid prior to delivery into the patient.

It would be preferable to have a system that, by its simplicity and use of low cost components, allowed for the disposal of the components that might otherwise require sterilization after use. If such simplicity could be combined with the versatility described and identified above, namely the ability to dispense multiple fluids in conjunction with multiple configuration nozzles, then most if not all of the problems associated with the prior art could thereby be addressed.

### SUMMARY OF THE INVENTION

In fulfillment of the above and other objectives the preset invention provides a system for the dispensing of surgical irrigation fluids that includes a hand gripped, hand operated dispensing unit connected through a single continuous length of multi-tube or multi-lumen conduit to a mobile reservoir cart capable of being positioned immediately adjacent the surgical site. The handheld pistol grip or pencil grip dispensing unit is constructed of low cost materials and incorporates no electrical components or parts. The conduit system extending from the fluid reservoirs in the reservoir cart to the handheld dispensing unit are uninterrupted in order to provide a sealed flow delivery of the irrigating fluids to the handheld dispensing unit. Flow control through these uninterrupted conduits is carried out by way of a trigger positioned on the handheld dispensing unit, as well as a plurality of on/off valves, individually selectable with each of the fluid tubes at the handheld unit. The handheld unit further includes a nozzle connector incorporating a tubing manifold that combines the three or more fluid flow conduits into a single flow nozzle. A suction tube extends in parallel arrangement out from the nozzle and passes back through the handheld unit to a vacuum or suction source within the hospital or medical facility. A variety of nozzles are provided with the system to vary the manner in which the irrigation fluids may be dispensed into a surgical site. These nozzles facilitate a number of different procedures associated with typical surgical operation including flushing and debriding.

The mobile reservoir unit of the system of the present invention includes a number of disposable reservoirs containing a variety of irrigation fluids required by the system within a cart incorporating caster wheels, such as may be moved to a position adjacent the surgical/operating table. The disposable reservoirs are modular in nature and may be quickly positioned in place within the mobile cart and connected to the tubing connectors that extend to the handheld unit. A heating unit may optionally be positioned adjacent the tubes flowing from the reservoirs to elevate the temperature of the fluid to an appropriate level. A specially designed rotary tube pump allows for the placement and removal of a closed section of fluid tubing in conjunction with the pump next to the dry roller system thereof. Such a configuration provides a nearly continuous flow of irrigation fluid at a variable rate, and is capable of continuous operation even in the face of back pressure brought about by momentary or long term stoppage of the flow by the physician at the handheld unit.

### BRIEF DESCRIPTION OF THE DRAWINGS

FIG. 1 is a schematic view of the primary components of the system of the present invention, including the handheld dispenser, the connection tubing, and the mobile reservoir unit.

FIG. 2 is a perspective view of one embodiment of the handheld dispensing component (detail) and the mobile cart unit of the system of the present invention.

FIGS. 3A-3C are detailed side, top, and front plan views (respectively) of the handheld dispensing component of the system of the present invention shown with a first embodiment of a nozzle assembly.

FIGS. 4A-4C are detailed side, top, and front plan views (respectively) of the handheld dispensing component of the system of the present invention shown with a second embodiment of a nozzle assembly.

FIG. 5A is a detailed partial cross-sectional view of the interior configuration of the handheld dispensing unit, showing the internal pathways of the associated tubing sections.

FIG. 5B is a detailed bottom plan view (looking up into the handle) of the handheld dispensing unit, showing the internal on/off valves associated with the tubing sections.

FIG. 6 is a detailed perspective view of the tubing manifold component of the handheld dispensing unit of the system of the present invention.

FIG. 7 is a detailed perspective view of the on/off valve block component of the handheld dispensing unit of the system of the present invention.

FIG. 8 is an exploded side plan view of a brush tool nozzle assembly associated with the embodiment of the handheld unit of the present invention shown in FIGS. 4A-4C.

FIG. 9 is a detailed interior back panel view of the components associated with the mobile reservoir unit of the system of the present invention.

FIG. 10 is a detailed perspective view of a typical tubing pump component used in conjunction with the mobile reservoir unit of the system of the present invention.

FIG. 11 is a detailed perspective view of a typical reservoir cap and vent tube assembly used in conjunction with the mobile reservoir unit of the present invention.

FIG. 12 is a detailed partial cross-sectional view of the interior configuration of an alternate preferred embodiment of the handheld dispensing unit, showing the internal pathways of the associated tubing sections.

### DETAILED DESCRIPTION OF THE PREFERRED EMBODIMENTS

As summarized above, the present invention comprises a system for irrigating wound and surgical areas on a patient, by an attending physician in an emergency room or other health care environment. The system includes a portable module shaped to fit a physician's hand as with a pistol configuration or a pencil configuration, as well as a mobile cabinet module that retains the ancillary equipment needed to complete the functionality of the irrigation system. The system of the present invention allows an attending physician to irrigate during surgery with at least three different fluid solutions instead of the more typical single irrigation solution. The system regulates the pressure of the fluid being dispensed so that no tissue damage will occur during the process. The system also allows the physician to cleanse the wound of debris using a variety of nozzle attachments connected to the handheld unit. In addition, the invention allows the irrigation fluids to be warmed to increase patient comfort and the control of the fluids by the physician. Irrigation volumes can be regulated for further control of the surgical environment. The fluid reservoirs carried in the cabinet module of the present invention are of sufficient size that the unit is generally ready to be used and does not require frequent restocking. The connections to the various fluid reservoirs are maintained in a sterile state.

The variety of nozzles associated with the handheld unit allows the physician to scrub, spray, gross irrigate, or fine irrigate the wound without releasing the irrigation gun. A large number of the components of this system are disposable, thus eliminating the need for disinfecting or otherwise sanitizing reusable components. All of the few reusable components in the system are easily and at times, automatically, disinfected through the operation of the system. The majority of the components in the system that might need to be sterile are manufactured from less costly materials that can be used once and then cost effectively disposed of. This is accomplished in part by the maintenance of a continuous, uninterrupted, and sealed fluid flow path from each of the irrigating fluid reservoirs through to the nozzle assembly of the handheld unit. Every component along this path that comes in contact with the fluid and/or the surgical environment is disposable. All other components in the system are isolated from the fluid and the surgical environment.

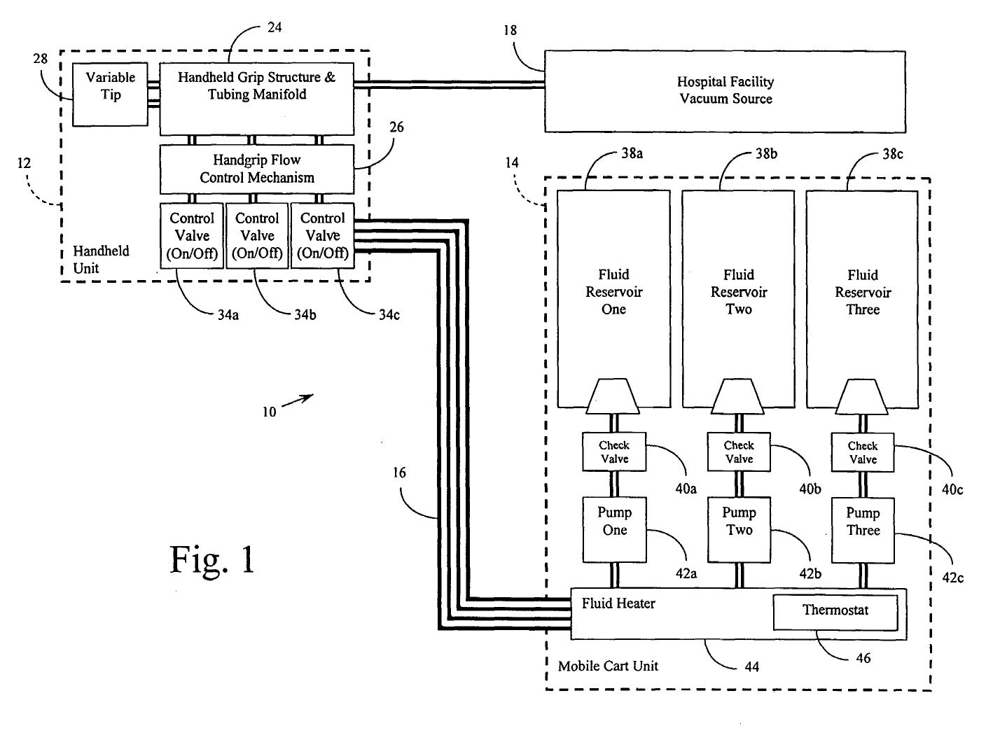

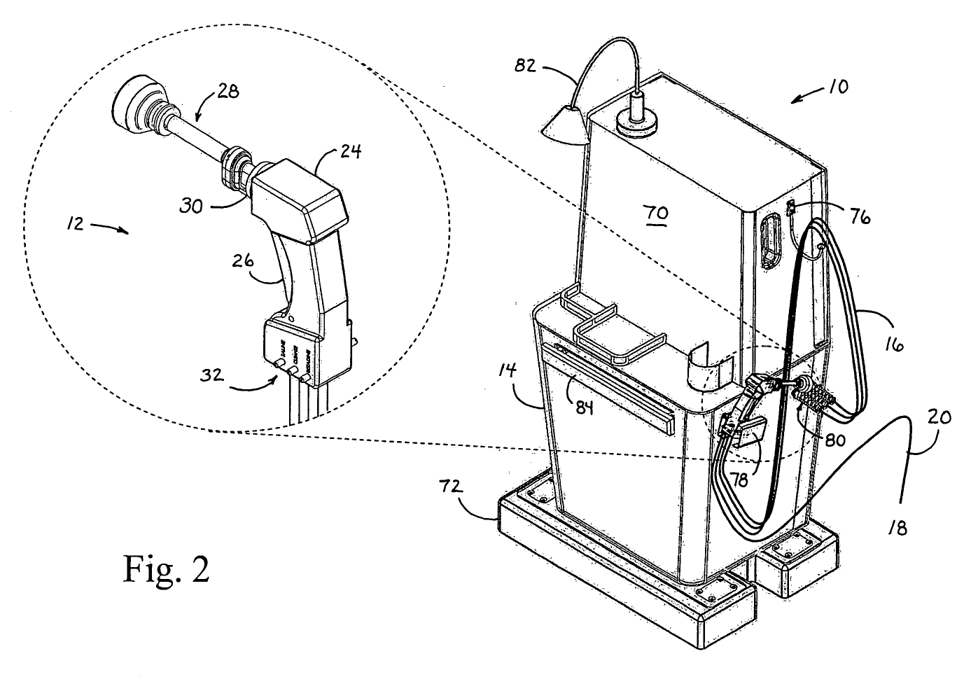

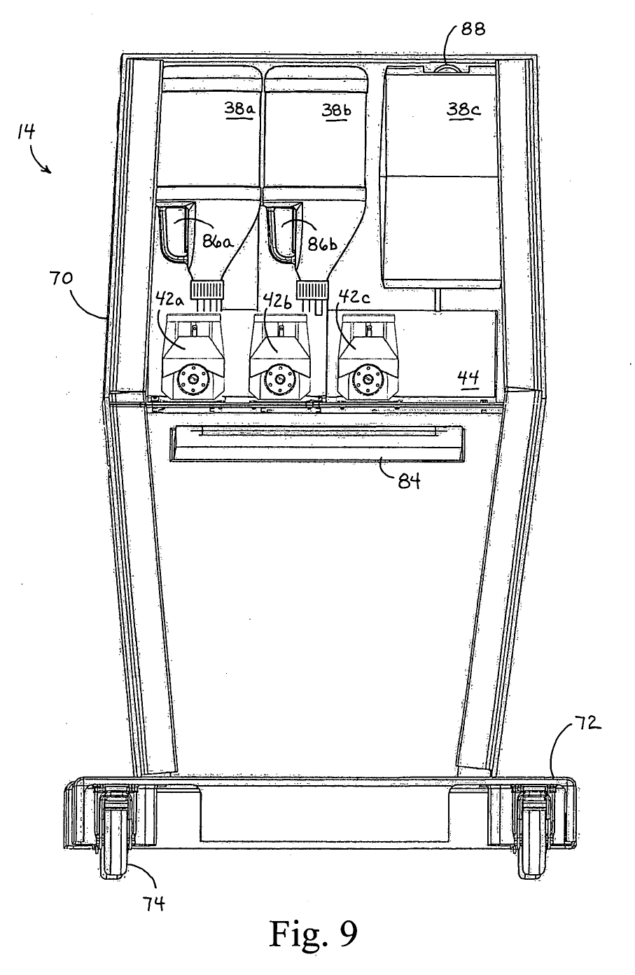

Reference is made first to FIG. 1 for a general description of the overall surgical irrigation system 10 of the present invention. FIG. 1 discloses the two primary components of the system and the array of tubing conduits that connect those two primary components together. The first primary component of the system is mobile reservoir cart unit 14 that provides the source of the fluids for the irrigation process. This mobile unit 14 comprises a cart positioned on a plurality of caster wheels (see FIGS. 2 and 9) that allow the entire unit to be easily moved to a position adjacent the operating table. The interior of the upright mobile cart unit 14 (the structure of which is described in more detail below) includes the various removable and disposable fluid reservoirs 38a, 38b, and 38c, along with a plurality of rotary pumps 42a, 42b, and 42c, one associated with each of the reservoirs. A fluid heater 44 and thermostat 46 (again described in more detail below) are also incorporated inside the mobile reservoir cart unit 14 as shown in FIG. 1. On the exterior of the reservoir cart unit are a number of features (not shown in FIG. 1) that facilitate the use of the system by the attending physician. These include a holster and other means for retaining the handheld component of the system and the associated tubing, when not in use (see FIG. 2).

Extending from the mobile reservoir cart unit 14 are a plurality of conduits, i.e., multi-lumen connection tubing 16, that extend directly from each of the fluid reservoirs 38a, 38b, and 38c, through check valves 40a, 40b, and 40c, and through rotary pumps 42a, 42b, and 42c, directly to the handheld dispensing unit 12 without interruption. As indicated above, an important feature of the present invention is the continuous multi-lumen connection tubing 16 that extends from the individual disposable fluid reservoirs all the way to the handheld grip structure and tubing manifold 24 near the tip or nozzle 28 of the handheld unit 12. In this uninterrupted manner, the opportunity for contamination of the fluid is minimized or nearly eliminated. This construction of the delivery conduits also facilitates the rapid and easy disposal of these inexpensive components of the system after use.

The second primary component of the system of the present invention as shown in FIG. 1 is the handheld dispensing unit 12 at the delivery end of the irrigation fluid multi-lumen connection tubing 16 conduits. Within this handheld unit 12 (and described in more detail below) are the mechanisms necessary to permit the attending physician to control the flow of the various fluids through the unit to the nozzle for dispensing into the surgical site. At the same time the handheld unit 12 provides the means to control suction of the various fluids through connection with the hospital facility vacuum source 18. These components include a handgrip flow control mechanism 26 that allows the physician to vary the flow according to the degree to which the trigger of the handheld unit is squeezed. Also associated with the handheld unit are a number of on/off valves 34a, 34b, and 34c. These valves are manipulable by the physician with one hand to control the on/off flow of the individual fluids through the device. As indicated above, the irrigation flow conduits extend entirely through the handheld unit 12 uninterrupted to a tubing manifold 24 positioned adjacent the interchangeable variable tip 28 at the outlet of the handheld unit 12. Every manner of control for the flow of fluid through the device is achieved without interrupting the conduits into separate sections. In other words, both the on/off flow control and the trigger grip flow control are accomplished by squeezing the tubing into a configuration that restricts or releases the flow of the irrigation fluid.

A variety of interchangeable nozzles may be positioned on the variable tip connector 28 shown on the handheld unit 12 in FIG. 1 in a manner that allows the physician to choose among a number of different functions for the irrigating fluid. Suction is provided through the suction nozzle, again located on the interchangeable variable tip connector 28 of the handheld unit 12.

FIG. 2 provides perspective views of the surgical irrigation system 10 and a close-up view of the handheld dispensing unit 12 of the present invention. The mobile reservoir cart unit 14 is shown with the multi-lumen connection tubing 16 looped over a tubing hanger 76 located on the side of the cabinet enclosure 70. The mobile reservoir cart unit 14 is positioned on and supported by a caster platform 72. Also integrated as external features on the cabinet enclosure 70 is lamp 82 and tray table 84 for ease of use during surgical or medical procedures. Various shelf surface compartments are also provided on the exterior of mobile reservoir cart unit 14.

Continuing reference to FIG. 2, the proximal ends of the multi-lumen connection tubing 16 are reinforced with strain relief coils 80 at the entrance to the cabinet enclosure 70. Also joining the connection tubing 16 is the vacuum source tubing 20 which is connected to the hospital facility vacuum source 18. As shown in FIG. 2, the handheld dispensing unit 12 may be conveniently positioned on the cabinet holster 78 when not in use.

The handheld dispensing unit 12 is shown in greater detail in the enlarged portion of FIG. 2. The handheld unit body structure 24 incorporates a pistol grip (in this preferred embodiment) flow control 26, an on/off control valve assembly 32, and a nozzle tip connector 30. An interchangeable nozzle tip 28 is positioned on the nozzle tip connector 30 (in this case a flushing probe assembly is attached).

In the detailed view in FIG. 2, the manner in which the plurality of irrigation fluid tubes enters the handheld unit is seen. The on/off valves associated with selecting the flow of irrigation fluid from among the three separate tubing conduits is also seen at the base of the handle of the pistol grip structured handheld unit. In this manner, the physician may manipulate the on/off valves associated with the flow control by simply pressing on one side or the other of the handle component of the handheld unit, thus engaging or releasing a crimping valve interior to the unit surrounding the individual flow tubes (the details of which are described below).

The pistol grip flow control 26 associated with the handheld unit body structure 24 is a movable trigger that extends into the unit in a manner that effectively crimps the tubing and thereby varies the flow of fluids through each of the three tubes that extend from their point of entry into the handheld unit up to the manifold adjacent the nozzle tip connector 30. In the view of FIG. 2, the nozzle tip connector 30 is shown positioned at an upper end of the handheld unit body structure 24, and is configured to receive interchangeable nozzle tips 28 which, in addition to a dispensing nozzle, incorporate a nozzle associated with the suctioned removal of fluid from the surgical area.

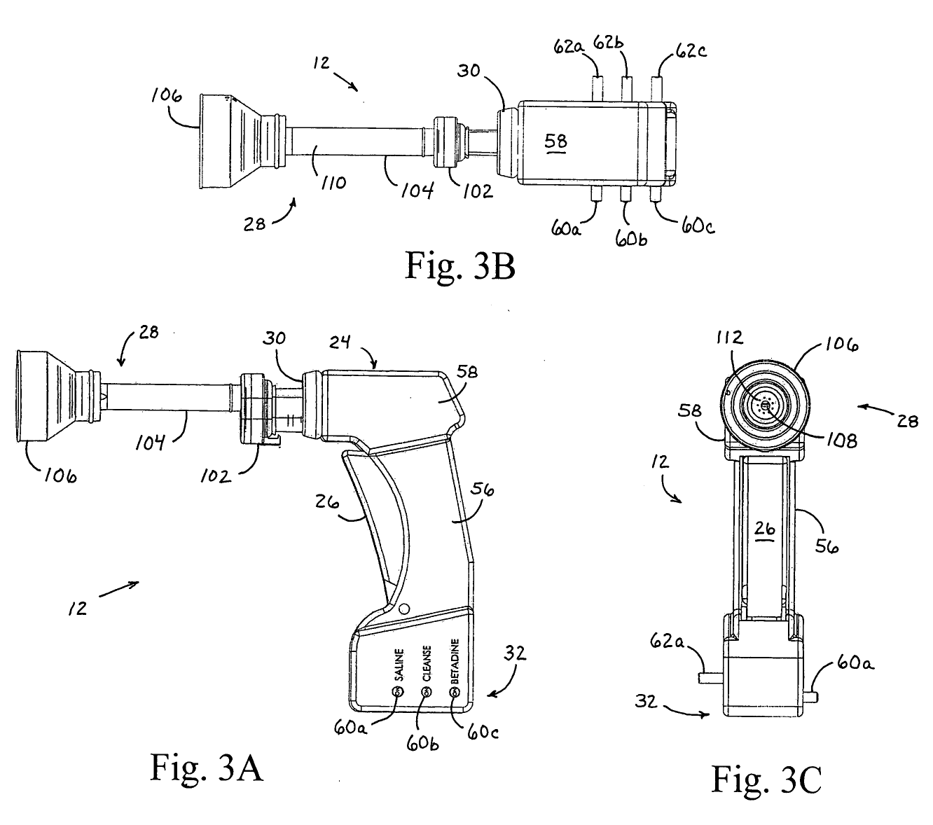

FIGS. 3A-3C are a detailed side, top, and front views (respectively) of the handheld dispensing unit 12 of the present invention showing the handheld unit body structure 24, the control valve assembly 32, and a first type of interchangeable nozzle tip 28. The handheld unit body structure 24 includes the handheld unit handle 56, the pistol grip flow control 26, the handheld unit barrel 58, and the nozzle tip connector 30. The control valve assembly 32 (which is primarily located within the handle structure of the unit) extends control valve “on” posts 60a, 60b, and 60c out from the handle for on/off control of the flow of fluids into the handheld unit of the system. The interchangeable nozzle tip 28 in this configuration (FIG. 3A) further includes adaptor 102, flushing probe barrel 104, and flushing probe cup 106.

FIG. 3B is a top plan view of the handheld dispensing unit 12 showing the manner in which the individual on/off valves (exposed as valve “on” posts 60a, 60b, and 60c, and valve “off” posts 62a, 62b, and 62c) associated with entry of the irrigation fluid flow into the unit 12. These valve on/off control posts are shown positioned on either side of the base of the handle component 56 of the handheld unit 12. In this view, the three push button control mechanisms for operating the internal crimping valves that regulate the on/off flow of the fluids through the discreet tubing components can be seen. The interchangeable nozzle tip 28 seen in this view includes flushing probe barrel 104, which as described below, coaxially integrates flushing probe discharge tube 110 around flushing probe suction tube 108 (not seen in this view). Alternate nozzle tips 28 may reverse the arrangement of the coaxial suction and discharge tubes, which reversal is accommodated by appropriately turning (180°) adaptor 102 to reverse the connection to the internal ports of nozzle tip connector 30.

FIG. 3C is a front view of the handheld dispensing unit 12 showing the handheld unit barrel 58, handheld unit handle 56, and the pistol grip flow control 26. This view of the control valve assembly 32 displays one pair of on/off control valve posts with “on” post 60a shown and “off” post 62a shown. Within flushing probe cup 106 of the interchangeable nozzle assembly 28, a plurality of aperture outlets comprising the flushing probe nozzle 112 are shown encircling the flushing probe suction tube 108. In this manner, a flushing spray of irrigation fluid may be directed within the confines of the cup 106 from nozzle 112 to be suctioned back again after contact with the wound tissue or the like, into suction tube 108.

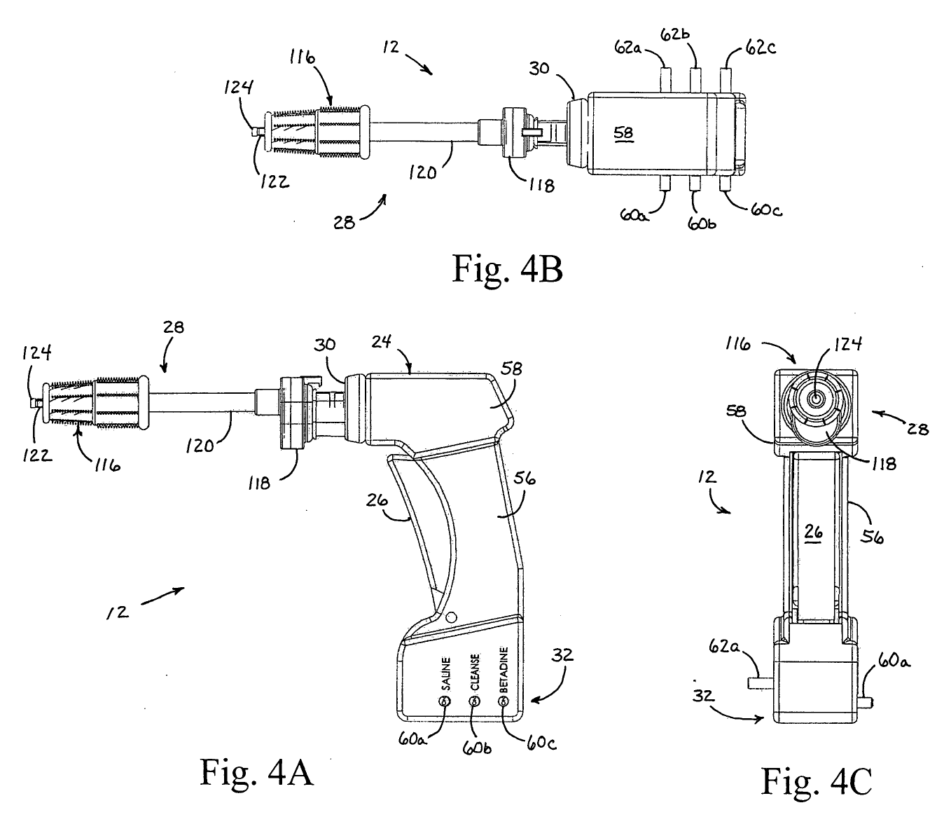

In the embodiment shown in FIGS. 4A, 4B, and 4C an alternate nozzle tip 28 is positioned on the nozzle tip connector 30 in a manner to show the different types of nozzles (and their functionality) capable of being used in conjunction with the handheld dispensing unit 12 of the present invention. As shown in FIGS. 4A, 4B, and 4C the alternate nozzle tip 28 may have a brush tool rather than the flushing probe design described above. In the brush tool configuration, the brush tool nose piece 118 connects through adaptor 102 to nozzle tip connector 30. As described following, this alternate nozzle assembly reverses the discharge and suction tubes from the coaxial arrangement described above. Adaptor 102 is therefore reversed (rotated 180°) in its orientation to appropriately direct the positive fluid flow and the return suction flow.

From this nose piece 118 extends brush tool suction tube 120 of the brush tool assembly 116. The brush tool assembly 116 further includes the brush tool head 126 having a brush tool nozzle 124 and a brush tool discharge tube 122. As indicated above, discharge tube 122 is coaxially situated within suction tube 120. The discharge of irrigating fluids therefore occurs through the center of this arrangement with the return flow occurring (through brush tool head 126) in a concentric ring around the discharge.

The size of the handheld unit is of course dictated by the size of the human hand, to allow for easy manipulation, not only of the trigger flow control, but also the on/off valve flow control and the rapid and simple interchange of a variety of nozzle configurations. While the profile of the handheld unit shown in FIGS. 2, 3A-3C, and 4A-4C, is a pistol grip configuration, the same functionality can be achieved by use of a pencil grip type configuration whereby the on/off valve structures might still be incorporated into the base of the unit (the area in which the tubing conduits enter the handheld unit) and the trigger might be positioned on top of the unit in a manner whereby the physician's hand might squeeze the barrel of the pencil-shaped dispenser to regulate the flow of fluids there through. The nozzle connector and associated interchangeable nozzles could likely take on the same configuration as in the pistol grip structure described above.

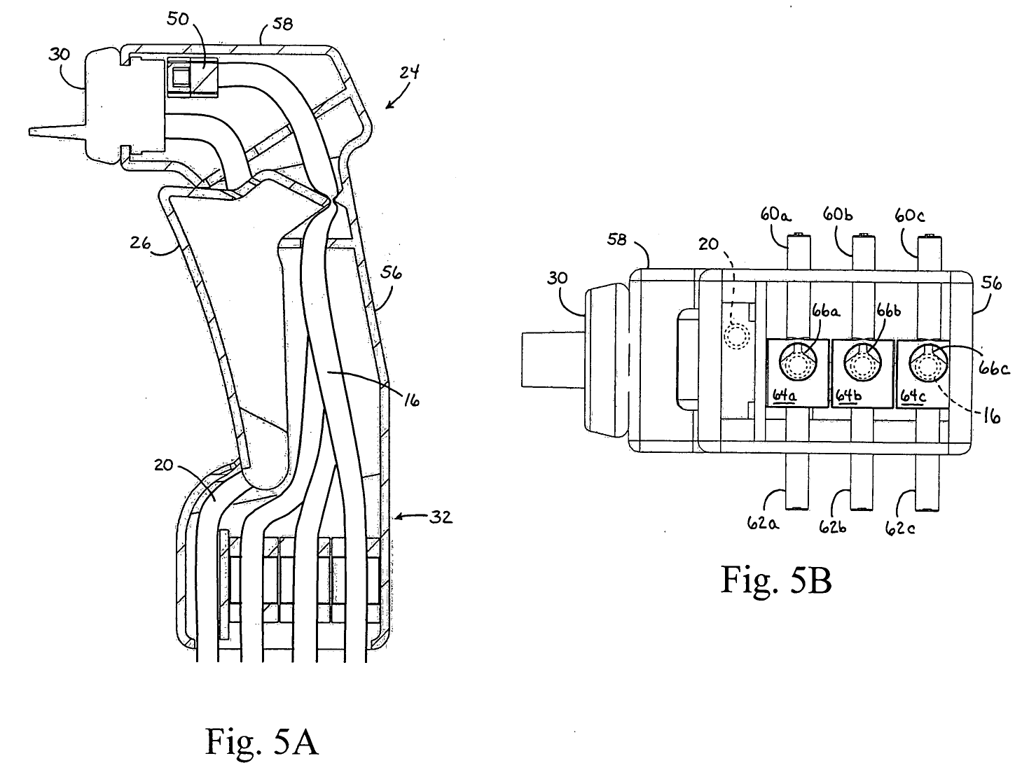

Reference is now made to FIGS. 5A and 5B for a detailed description of the internal components of a first preferred embodiment of the handheld unit body structure 24 of the irrigation system 10 of the present invention. FIG. 5A is a side cutaway view wherein it becomes clear the manner in which the multi-lumen connection tubing 16, which connects back to the reservoir cart component of the present invention, extends into the handheld unit handle 56 of the handheld unit through the three on/off valves and up to a point where each of the three conduits connects to the tubing manifold 50. At this final point, the tubing manifold 50 first joins the three previously discrete sections of tubing (discrete lumens) into a single dispensing conduit that extends out from the handheld unit, through the nozzle tip connector 30 into the interchangeable nozzle (not shown in this view). Along the way from the base of the handheld unit to the tubing manifold, the pistol grip flow control 26 of the device engages the three tubing conduits placed parallel to each other in a manner that allows the physician to variably restrict the flow of fluid through the entire bank of conduits prior to the flow arriving at the tubing manifold 50.

When the pistol grip flow control 26 is released, the trigger is internally removed from the tubing conduits 16 in a manner that allows the unrestricted flow from the on/off valves through to the tubing manifold 50. When squeezed, the trigger constricts all three sections of tubing in a manner that variably limits the fluid flow through each of the three tubing sections. Of course, this fluid flow is initially determined by the status of the on/off control valves 34a, 34b, and 34c, as evidenced by the position of control valve posts 60a-60c and 62a-62c positioned at the base of the handle 56 of the handheld dispensing unit as seen in the bottom plan view of FIG. 5B. Also visible in this view are the control valve blocks 64a, 64b, and 64c which are an integral part of the unit handle 56, as well as the control valve pinch channels 66a, 66b, and 66c through which the various sections of the multi-lumen connection tubing 16 extend. In operation, for example, control valve on post 60a is pushed toward the unit handle 56, shifting valve block 64a, and therefore tubing 16, away from pinch channel 66a and into the open section of the hole in valve block 64a, thus allowing the fluid to flow through the tubing 16. Valve blocks 64a-64c are structured to slide (side to side) within similarly shaped guides molded into the interior walls of the handle section 56 of handheld unit body structure 24. Likewise, valve pinch channels 66a-66c are formed in a fixed manner in the interior structure of the molded handle such that as the physician pushes on one of the extending valve posts the valve block slides and shifts the position of the section of tubing held captive within the valve block. In this manner, the tubing section is either forced into the V-shaped channel, thus cutting off the flow of fluid through the tubing, or is directed out from the V-shaped channel, thus re-establishing a flow of fluid through the tubing.

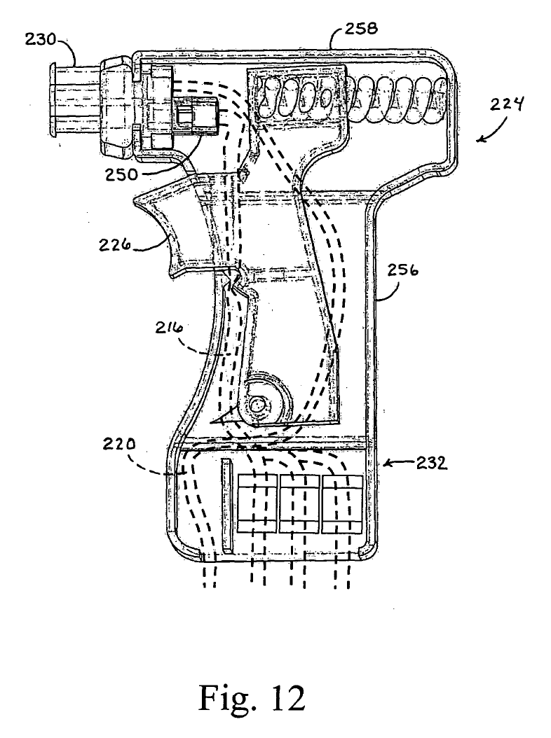

In all likelihood, and in typical use, only one of the three irrigation fluids might be selected at a given point in the surgical procedure, such that the trigger mechanism 26 need only act against the pressurized fluid flow of a single one of the three tubing sections. Nonetheless it is possible, in the configuration shown, for the trigger mechanism 26 to adequately restrict a flow of fluid through all of the conduits simultaneously. Those skilled in the art will recognize that although the first preferred embodiment shown represents a “normally on” flow with compression of the trigger mechanism 26 serving to inhibit such flow, simple modifications to the design would permit implementation of the reverse with a “normally off” state and the initiation of fluid flow with the compression of the trigger mechanism. This alternate preferred embodiment structure is described below in conjunction with FIG. 12.

FIG. 5A once again, is a detailed cross-sectional view of the interior of handheld unit body structure 24 of the system of the present invention. In this view, the tubing manifold 50 and the manner in which it connects the three discreet sections of tubing (combined as multi-lumen connection tubing 16) into a single dispensing port can be seen. Fixed in position adjacent the nozzle connector 30 in the handheld unit, tubing manifold 50 simply connects the flow conduits from each of the three discreet tubing sections into a single dispensing conduit that flows out through the nozzle connector 30 and into the interchangeable nozzle assembly 28 (not shown in this view) by way of adaptor 102 (also not seen in this view). Depending upon the specific nozzle assembly selected, the fluid flows outward from the nozzle into the surgical area under the control of the attending physician. Shown more clearly in FIGS. 3C and 4C, the suction nozzle can be seen positioned adjacent the dispensing nozzle in the handheld unit. This positioning, and the control of the flow of the irrigation fluids by the physician, allow for the alternate or simultaneous dispensing and suction of irrigation fluids into and from the surgical area.

It is anticipated that the entire structure associated with the handheld unit, extending back to the point of conduit attachment to the fluid reservoirs, may be manufactured complete without need for on-site assembly beyond attachment to the reservoirs. In other words, the pathways of the tubing internal to the handheld unit, from the manifold out, may be manufactured in place, and may be disposed of after use. Bonding of the ends of the tubing into the manifold may be accomplished by any of a number of methods for bonding polymer based tubing to plastics and the like.

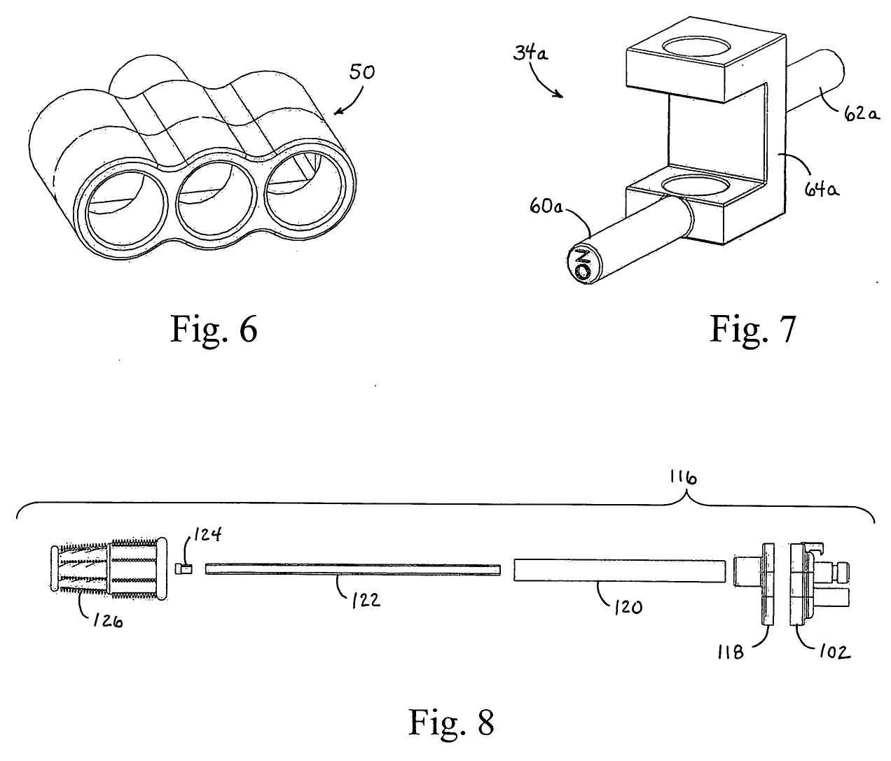

FIG. 6 is a perspective view of the tubing manifold component 50 of the handheld unit of the system of the present invention showing in detail the manner in which the three separate lumens (typical) are combined into one lumen at the point of exiting the body structure of the handheld unit, and passing into the various nozzle assemblies (by way of the appropriately positioned adaptor). As indicated above, manifold 50 is the terminal end of the uninterrupted length of multi-lumen (or multi-tube) conduit that extends from the irrigation fluid reservoirs in the mobile reservoir cart unit of the system.

FIG. 7 is a detailed perspective view of a typical (one of three in the preferred embodiment) on/off valve block 64a component of the control valve 34a of the handheld unit 12 of the system of the present invention, showing the manner in which a length of tubing may pass there through and be held sufficiently fixed as to be forced into or out from the V-channel pinch plate 66a as described above. Extension posts 60a (as an example) and 62a are positioned to extend out through aligned apertures in the walls of the handle of the handheld unit to allow the user to push the appropriate post to direct the tubing into or out from the V-channel as desired. Again, the handheld unit would be manufactured with the appropriate ends of the tubing section threaded through the apertures in the on/off valve blocks before being secured in to manifold 50 as described above.

FIG. 8 is an exploded side plan view of a brush tool assembly 116 associated with the second embodiment of the handheld unit of the present invention (shown in detail in FIGS. 4A-4B) presented here in an exploded configuration primarily to demonstrate the manner in which the discharge tube 122 and the suction tube 120 for the various nozzle assemblies may be concentrically (coaxially) arranged. Once again, the arrangement of these two tubes may be reversed by simply rotating the adaptor 102 to re-align the appropriate ports on nozzle tip connector 30. Various types of nozzle assemblies will benefit from one or the other arrangement of coaxially aligned tubes.

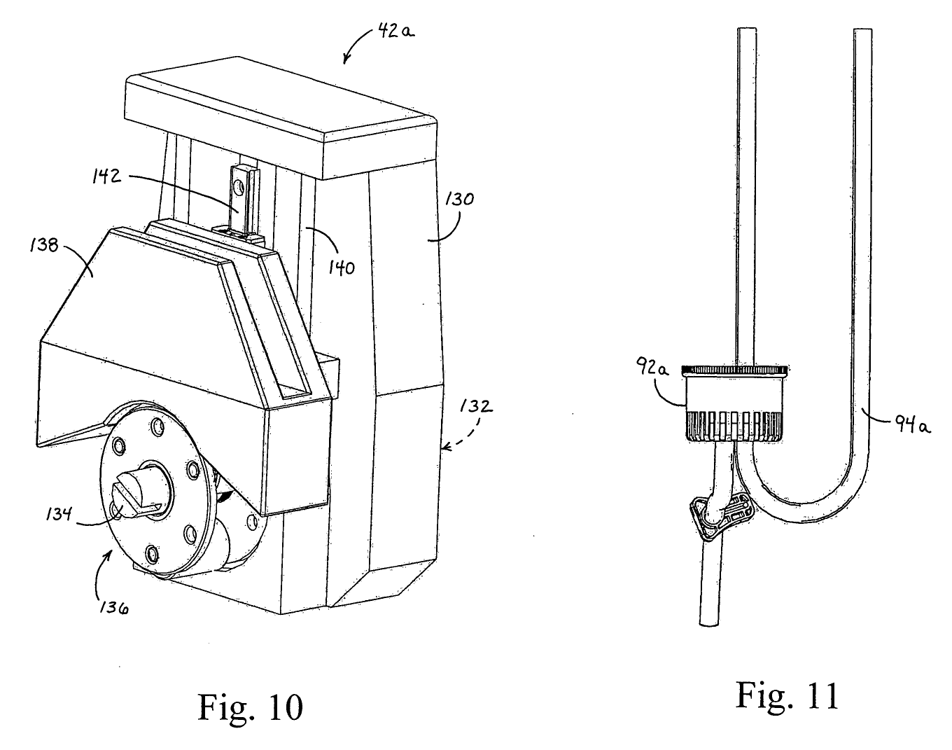

FIG. 9 is a detailed interior (back panel) view of the internal components associated with the mobile reservoir unit 14 of the present invention which shows the actual physical arrangement of these various components described functionally above with respect to FIG. 1. The array of tubing pumps 42a-42c are shown and described in greater detail in FIG. 10. The structure of cart unit 14 is designed to provide easy access to the replaceable fluid reservoirs 38a-38c as well as the multi-lumen (multi-tube) conduit sections that extend from the reservoirs, and further to provide portability through the use of casters 74 positioned in caster platform 72. In the preferred embodiment shown in FIG. 9, reservoirs 38a and 38b are configured as one gallon bottles (such as might contain a betadine solution or a water based cleansing solution) supported in an inverted position by reservoir support brackets 86a and 86b respectively. Reservoir 38c is, in the embodiment shown, configured as a typical IV bag container (such as might contain a saline solution) and is supported within the cabinet enclosure 70 by reservoir support hanger 88.

The process of installing the disposable portions of the system into the mobile reservoir cart unit 14 generally starts by removing a shipping lid from the bottle type containers and replacing it with the reservoir lid/vent tube assembly shown and described below with FIG. 11. These lid/vent tube assemblies are incorporated onto the end of the multi-lumen (multi-tube) conduit and form part of the disposable components of the system. Attachment of a similar connector to the outlet of the IV bag type reservoir is also carried out. Each of the reservoirs is then placed on their associated support brackets. The tube sections of the multi-lumen conduit (not shown for clarity in FIG. 9) are then passed under each of the tubing clamps associated with the rotary tubing pumps 42a-42c and in this manner are engaged to initiate fluid flow from the reservoirs upon activation of the pumps. The tubing sections thereafter pass out of the cabinet enclosure as described above with respect to FIG. 2.

Heater 44 (and its associated thermostatic control) may be positioned on either side of the rotary tubing pumps 42a-42c in the fluid flow path. The effort is to elevated the temperature of the fluids to a warm (room temperature or slightly above) level for the purpose of both patient and physician comfort. Minimal contact between a low current electrical heating element and the tubing sections, either before or after the rotary tubing pumps, is typically sufficient to achieve such low level warming.

FIG. 10 provides perspective detail on a typical one of the rotary tubing pumps 42a (for example). This special tubing pump design provides more than adequate fluid flow for irrigation purposes while still being operable against an intentional stoppage of flow by action of a downstream valve. In other words, even if one of the control valve elements of the handheld unit of the system restricts or stops the flow of fluid within the tubing, the rotary tubing pump may continue to operate (rotate) without undue strain on the pump or the tubing positioned within it. Tubing pump 42a shown in FIG. 10 includes pump body assembly 130 which supports and positions drive motor 132 (hidden in this perspective view) and drive shaft 134.

Drive shaft 134 extends through to engage drive roller assembly 136 which, in the preferred embodiment includes three free-rotating rollers that act in planetary movement about the axis of rotation of drive shaft 134. Tubing clamp 138 is positioned and configured to hold a section of tubing against drive roller assembly 136 in a manner that effects a flow of liquid within the tubing section directed by the progressive compression of the tubing by the rollers. Tubing clamp 138 is preferenced against roller assembly 136 by a pair of springs (not shown for clarity) that ride on spring guides 140 (two in the preferred embodiment shown). The section of tubing may be inserted into the space between tubing clamp 138 and roller assembly 136 by lifting tubing clamp 138 against the force of the springs, inserting the tubing, and releasing the clamp. Tubing clamp 138 moves apart from and towards roller assembly 136 by sliding on track 142 positioned on pump body assembly 130. The flow rate generated by pump 42a may be modified by changing the force with which the tubing clamp springs preference the clamp against the roller assembly. Various mechanisms for tightening (or loosening) the springs, know in the art, may be used to vary this force.

Reference is now made to FIG. 11 for a detailed perspective view of a typical reservoir cap 92a and vent tube 94a assembly associated with the mobile reservoir unit of the present invention. As described above, installation of the multi-lumen (multi-tube) conduit component of the present system onto the reservoirs involves replacing a shipping cap with the disclosed ventilated cap. This arrangement allows fluid to be drawn from the inverted bottle reservoirs while replacement air is drawn into the bottles by way of the vent tube 94a. The IV bag structured reservoir, of course, is designed to collapse as the fluid is withdrawn and therefore does not require ventilation.

Reference is now made to FIG. 12 for a detailed description of the internal components of an alternate embodiment of the handheld unit body structure 224, of the irrigation system of the present invention. FIG. 12 is a side cutaway view that shows the manner in which multi-lumen connection tubing 216, which connects back to the reservoir cart component of the present invention, extends into an alternate handheld unit handle 256 configured to provide operation of a handheld unit in a “normally-off” configuration. Once again, multi-lumen connection tubing 216 connects to the reservoir cart through the handheld unit handle 256 of the handheld unit through the three (3) on/off valves (as in the first preferred embodiment) and up toga point where each of the three (3) conduits connects to the tubing manifold 250. At this point the tubing manifold 250 first joins the three (3) previously discrete sections of tubing (discrete lumens) into a single dispensing conduit that extends out from the handheld unit, through the nozzle tip connector 230 into the interchangeable nozzle (not shown in this view). Along the way from the base of the handheld unit to the tubing manifold, the pistol grip flow control 226 of this alternate embodiment of the device engages the three (3) tubing conduits placed parallel to each other in a manner that allows the physician to variably direct the flow of fluid through the entire bank of conduits prior to the flow arriving at the tubing manifold 250.

When the pistol grip flow control 226 is released, spring 227 preferences the tubing conduits 216 in a manner that restricts the flow from the on/off valves through to the tubing manifold 250. When squeezed, the trigger releases all three (3) sections of tubing in a manner that variably allows the flow of fluid through each of the three (3) tubing sections. Of course, this fluid flow is still initially determined by the status of the on/off control valves (not shown in this view) as described above in conjunction with the first preferred embodiment of the invention.

FIG. 12 once again is a detailed cross-sectional view of the interior of handheld unit and body structure 224 of the system of the present invention. In this view, the tubing manifold 250 and the manner in which it connects to the three (3) discrete sections of tubing (combined as multi-lumen connection tubing 216) into a single dispensing port can be seen. It is anticipated that in the configuration shown as an alternate preferred embodiment in FIG. 12, tubing manifold 216 might comprise a thinner walled or smaller diameter conduit that reduces the force that must be exerted by the spring 227 in order to fully compress the conduit and stop the flow of fluid. The vacuum line 220 shown in FIG. 12 is essentially the same as that described above with the first preferred embodiment. Operation of the alternate embodiment shown in FIG. 2 comprises the physician compressing the trigger (toward the back of the handle unit) to work against the force of the spring 227 and thereby release compression at the pinch point on the tubing in order to allow the flow of fluid there through. Various voids in the handle unit itself are provided to allow the routing of the conduits (both the vacuum conduit and the fluid flow conduit) to either side of the trigger component 226 of the pistol grip handle 224 in a manner that allows operation of the pistol grip without obstruction from the tubing passing to either side.

Although the present invention has been described in terms of the foregoing preferred embodiments, this description has been provided by way of explanation only, and is not intended to be construed as a limitation of the invention. Those skilled in the art will recognize modifications of the present invention that might accommodate specific irrigation techniques or environmental requirements. Such modifications as to structure, orientation, geometry, and even materials, do not necessarily depart from the spirit and scope of the invention.

I claim:

1. A system for controllably delivering a plurality of distinct irrigation fluids to a patient undergoing medical treatment, the system comprising:
a mobile reservoir cart unit, the cart unit comprising a reservoir support structure, a plurality of fluid reservoirs, and at least one fluid pump;
a handheld dispensing unit, the dispensing unit comprising a graspable body structure, an interchangeable nozzle assembly, a multi-lumen manifold, a plurality of discrete on/off control valves, and a gross flow control mechanism; and
a continuous length of multi-lumen conduit connecting the plurality of fluid reservoirs in the cart unit to the multi-lumen manifold in the dispensing unit, wherein the continuous multi-lumen conduit is compressible to interrupt a flow of fluids there through.

2. The system of claim 1 wherein the at least one fluid pump comprises a plurality of fluid pumps, each pump associated with a single one of the fluid reservoirs.

3. The system of claim 1 wherein the at least one fluid pump comprises a tubing pump that directs a flow of fluids in the multi-lumen conduit by progressively compressing a point along a length of the conduit in the direction of desired flow.

4. The system of claim 2 wherein the plurality of fluid pumps each comprise a tubing pump that directs a flow of fluid in one lumen of the multi-lumen conduit by progressively compressing a point along a length of the lumen within the conduit in the direction of desired flow.

5. The system of claim 1 wherein the mobile reservoir cart unit further comprises a plurality of check valves positioned in association with the plurality of reservoirs to facilitate the maintenance of the flow of fluid out from the plurality of reservoirs.

6. The system of claim 1 wherein the mobile reservoir cart unit further comprises at least one heater for elevating the temperature of the irrigation fluids to a desired level as the fluids flow through the multi-lumen conduit.

7. The system of claim 1 wherein the continuous length of multi-lumen conduit comprises a plurality of continuous length, single lumen, tubing sections.

8. The system of claim 1 further comprising a suction conduit connecting the interchangeable nozzle assembly of the handheld unit with a remote vacuum source.

9. The system of claim 1 wherein the plurality of distinct irrigation fluids comprises at least one irrigation fluid selected from the group consisting of saline solution, betadine solution, and purified water based cleansing solution.

10. An apparatus for dispensing a plurality of distinct irrigation fluids from a plurality of fluid reservoirs, the fluids delivered under pressure to the apparatus through a plurality of lumens within at least one length of flexible tubing, the apparatus comprising:
a graspable body structure, sized and shaped to fit in the hand of a user;
an interchangeable nozzle assembly, removably attachable to the body structure and comprising at least one outlet nozzle for directing a flow of irrigation fluid there through;
a multi-lumen manifold positioned in association with the body structure and in fluid flow communication with the nozzle assembly, the manifold connecting and combining the plurality of lumens within the at least one length of flexible tubing;
a plurality of discrete on/off control valves, each one of the control valves associated with one of the plurality of lumens within the at least one length of flexible tubing, each control valve further comprising a mechanism for alternately pinching closed or releasing open, one of the plurality of lumens in the flexible tubing; and
a gross flow control mechanism positioned in association with the body structure and movable between a position pinching closed the plurality of lumens within the at least one length of flexible tubing, and a position releasing open the plurality of lumens.

11. The apparatus of claim 10 wherein the interchangeable nozzle assembly further comprises a flushing attachment, the flushing attachment comprising:
an adaptor for connecting the flushing attachment to the graspable body structure, the adaptor comprising a fluid dispensing port and a suction port;
a barrel connected to the adaptor, the barrel comprising a fluid dispensing tube and a suction tube, the suction tube positioned coaxially within the fluid dispensing tube;
a nozzle connected to the fluid dispensing tube of the barrel, the nozzle defining a plurality of apertures through which fluid may be directed and a central aperture through which suction may draw fluid back into the assembly; and
a cup shaped shroud positioned about the nozzle to limit an area over which the fluid is directed from the nozzle.

12. The apparatus of claim 10 wherein the interchangeable nozzle assembly further comprises a brush tool attachment, the brush tool attachment comprising:
an adaptor for connecting the brush tool attachment to the graspable body structure, the adaptor comprising a fluid dispensing port and a suction port;
a barrel connected to the adaptor, the barrel comprising a fluid dispensing tube and a suction tube, the fluid dispensing tube positioned coaxially within the suction tube;
a nozzle connected to the fluid dispensing tube of the barrel, the nozzle defining at least one aperture through which fluid may be directed; and
a brush shroud positioned about the nozzle and connected to the suction tube, the brush shroud comprising a plurality of apertures and a plurality of bristles to permit abrasive contact in conjunction with the dispensing of fluids.

13. The apparatus of claim 10 wherein the plurality of discrete on/off control valves, each comprise;
a moveable valve block defining an aperture through which passes a section of tubing defining one of the plurality of lumens;
a fixed valve plate defining a V-channel adjacent which passes the section of tubing passing through the moveable valve block;
a first post extending from the moveable valve block and positioned such that a force exerted against an end of the first post pushes the section of tubing out from the V-channel in the fixed valve plate; and
a second post extending from the moveable valve block and positioned such that a force exerted against an end of the second post pushes the section of tubing into the V-channel in the fixed valve plate.

14. The apparatus of claim 10 wherein the interchangeable nozzle assembly further comprises a suction port and the apparatus further comprises a section of suction tubing extending from the suction port through the graspable body structure of the apparatus.

15. The apparatus of claim 10 wherein the gross flow control mechanism further comprises a preferencing mechanism, the preferencing mechanism positioning the gross flow control mechanism in a position pinching closed the plurality of lumens within the at least one length of flexible tubing.

16. The apparatus of claim 10 wherein the gross flow control mechanism further comprises a preferencing mechanism, the preferencing mechanism positioning the gross flow control mechanism in a position releasing open the plurality of lumens within the at least one length of flexible tubing.

17. An apparatus for pumping fluid through a section of flexible tubing, the apparatus operable to generate fluid flow when little or no resistance to flow in the tubing exists and further operable to maintain a static fluid state when a level of resistance to flow in the tubing exists, the apparatus comprising:
a drive motor and drive shaft;
a drive roller assembly coaxially connected to the drive shaft and comprising a plurality of planetary rollers radially arrayed about a central axis of the drive roller and each individually rotatable about its own axis;
a tubing clamp movable between a position adjacent to and partially around the drive roller assembly and a position apart from the drive roller assembly; and
a pump frame assembly, the frame assembly retaining, supporting, and allowing for the rotation of, the drive motor and drive roller assembly, the pump frame assembly further slidably supporting the tubing clamp;
whereby fluid flow is generated within the section of flexible tubing when it is placed between the tubing clamp and the drive roller assembly and the drive motor is activated.

18. The apparatus of claim 17 further comprising at least one spring positioned between the pump frame assembly and the tubing clamp, the at least one spring preferencing the tubing clamp into a position adjacent to and partially around the drive roller assembly.

19. The apparatus of claim 18 further comprising an adjustment mechanism whereby a force with which the at least one spring preferences the tubing clamp into a position adjacent to and partially around the drive roller assembly, may be varied.

20. A system for controllably delivering at least three distinct irrigation fluids to a patient undergoing medical treatment, the system comprising:
a mobile reservoir cart unit, the cart unit comprising:
a reservoir support structure;
at least three fluid reservoirs;
at least three continuously operable fluid pumps; and
a fluid heater;
a handheld dispensing unit, the dispensing unit comprising:
a graspable body structure;
an interchangeable nozzle assembly, the nozzle assembly comprising:
an adaptor for attaching the nozzle assembly to the body structure;
a dispensing conduit;
a suction conduit;
a discharge nozzle connected to the dispensing conduit;
a multi-lumen manifold;
at least three discrete on/off control valves; and
a single flow control device positioned as a trigger mechanism;
a continuous length of multi-tube conduit connecting the at least three fluid reservoirs in the cart unit to the multi-lumen manifold in the dispensing unit, wherein the continuous multi-tube conduit is compressible to interrupt a flow of fluids; and
a length of suction tubing connecting the interchangeable nozzle assembly with an existing vacuum source.
